(19) United States
(12) Patent Application Publication
(10) Pub. No.: US 2019/0117123 A1
(43) Pub. Date: Apr. 25, 2019

(54) Systems to detect movement of a user

(71) Applicant: Trek Technology (S) Pte. Ltd., Singapore (SG)

(72) Inventor: Joon Yong Wayne TAN, Singapore (SG)

(73) Assignee: Trek Technology (S) Pte. Ltd., Singapore (SG)

(21) Appl. No.: 15/761,022

(22) PCT Filed: Sep. 16, 2016
(86) PCT No.: PCT/SG2016/050452

§ 371 Date: Mar. 16, 2018

(30) Foreign Application Priority Data
Sep. 16, 2015 (SG) 10201507707Y
Sep. 15, 2016 (SG) 10201607712V

(51) Int. Cl. A61B 5/11; A61B 5/117; A61B 5/00; G16H 40/67

(52) U.S. Cl. CPC A61B 5/1112; A61B 5/117; A61B 5/11; A61B 5/742; A61B 5/1123; A61B 2562/0247; A61B 5/6829; G16H 40/67; A61B 2560/0214; A61B 2560/0431; A61B 2562/0219; A61B 5/0022

(57) ABSTRACT

Systems, devices, and methods for detecting movement of a user, comprising a portable device, database, and controller, portable device including a sensor assembly and processor, the sensor assembly having a sensor configurable to perform a first measurement, the processor configurable to compare the first measurement to a threshold value, and also configurable to transmit a code when the processor determines, based on the compare, that the first measurement is greater than or equal to the threshold value, the controller being configurable to receive a communication signal having the code and the controller also configurable to obtain, from the received communication signal, the code, and further the controller also configurable to display, on a graphical display, a notification message which includes at least an identification of the portable device user.

### BACKGROUND

The present disclosure relates generally to systems, devices, controllers, and methods for detecting a movement of a user, and more specifically, to systems, devices, controllers, and methods for performing, among other things, detecting, identifying, verifying, measuring, and/or monitoring of a movement, non-movement, location, identity, and/or other attributes of one or more users.

With recent advances and improvements in medical science and technology, medical professionals continue to improve on their abilities to provide medical services to patients. Such medical services include diagnosing, treating, curing, and providing care for patients suffering from health-related problems and/or the elderly. Despite such advancements and improvements, certain problems arise, return, and/or continue to exist when diagnosing, treating, curing, and/or providing care for some patients suffering from certain health-related problems. Such patients may include those suffering from dementia and/or those patients who may aggravate, prolong, worsen, and/or create new health-related problems when they attempt to move, stand up, and/or walk.

### BRIEF SUMMARY

Conventional approaches to providing care for certain patients generally require caregivers to perform continuous monitoring of such patients. With recent advances in technology, certain solutions enable caregivers to perform monitoring of patients without the caregiver needing to always be present within the confines of the patient's room. For example, closed-circuit television (CCTV) technology has enabled caregivers to continue visual monitoring of patients from remote locations. While such solutions may allow caregivers to perform remote visual monitoring of patients under their care, such solutions will only be effective when caregivers are viewing the CCTV television display at a time when a needed situation arises.

Present example embodiments relate generally to systems, devices, controllers, and methods for performing, among other things, detecting, identifying, verifying, measuring, and/or monitoring of a movement, non-movement, location, identity, and/or other attributes of one or more users.

In an exemplary embodiment, a system for detecting a movement of one or more users is described. The system may comprise a portable device, a database, and a controller. The portable device may include a sensor assembly and a processor. The sensor assembly may have a sensor configurable to perform a first measurement. The processor may be configurable to compare the first measurement to a threshold value. The processor may also be configurable to transmit a code when the processor determines, based on the compare, that the first measurement is greater than or equal to the threshold value. The code may include an identification of the portable device and/or the user of the portable device. The database may be configurable to store information, including the code and user information associated with the code. The controller may be in communication with the database. The controller may be configurable to receive communication signals, including a first communication signal having the code. The controller may also be configurable to obtain, from the received first communication signal, the code. The controller may also be configurable to retrieve, from the database and based on the obtained code, the user information associated with the obtained code. The retrieved user information may include an identification of the user of the portable device. The controller may also be configurable to display, on a graphical display, a notification message. The notification message may include at least the retrieved identification of the user of the portable device.

In another exemplary embodiment, a portable device for detecting a movement of one or more users is described. The portable device may comprise a sensor assembly and a processor. The sensor assembly may include a sensor, the sensor configurable to perform a first measurement. The processor may be transitionable between an off state and an on state. The on state may be a state resulting when a predetermined action is performed on at least one portion of the sensor assembly. The off state may be a state resulting when the predetermined action is not performed on the at least one portion of the sensor assembly. The processor may be configurable to, responsive to the processor being in the on state, compare the first measurement to a threshold value. The processor may also be configurable to, responsive to the processor being in the on state, transmit a signal when the processor determines, based on the compare, that the first measurement is greater than or equal to the threshold value.

In another exemplary embodiment, a portable device for detecting a movement of one or more users is described. The portable device may comprise a sensor assembly and a processor. The sensor assembly may include a sensor. The sensor may be transitionable between an off state and an on state. The on state may be a state resulting when a predetermined action is performed on at least one portion of the sensor assembly. The off state may be a state resulting when the predetermined action is not performed on the at least one portion of the sensor assembly. The sensor may be configurable to, responsive to the sensor being in the on state, perform a first measurement. The processor may be in communication with the sensor assembly. The processor may be configurable to receive, from the sensor assembly, the first measurement. The processor may also be configurable to compare the first measurement to a threshold value. The processor may also be configurable to transmit a signal when the processor determines, based on the compare, that the first measurement is greater than or equal to the threshold value.

### BRIEF DESCRIPTION OF THE DRAWINGS

For a more complete understanding of the present disclosure, example embodiments, and their advantages, reference is now made to the following description taken in conjunction with the accompanying drawings, in which like reference numbers indicate like features, and:

FIG. 1 is an illustration of an example embodiment of a system for detecting a movement of one or more users;

FIG. 2 is a perspective illustration of an example embodiment of a portable device for detecting a movement of one or more users;

FIG. 3A is a top view illustration of an example embodiment of a portable device for detecting a movement of a user;

FIG. 3B is a cross-sectional view illustration of an example embodiment of a portable device for detecting a movement of a user;

FIG. 4A is a top view illustration of an example embodiment of a portable device for detecting a movement of a user;

FIG. 4B is a cross-sectional view illustration of an example embodiment of a portable device for detecting a movement of a user;

FIG. 5 is an illustration of an example embodiment of a system for detecting a movement of one or more users;

FIG. 6 is an illustration of an example embodiment of a system for detecting a movement of one or more users;

FIG. 7 is an illustration of another example embodiment of a system for detecting a movement of one or more users; and

FIG. 8 is an illustration of an example embodiment of a method for detecting a movement of one or more users.

Although similar reference numbers may be used to refer to similar elements in the figures for convenience, it can be appreciated that each of the various example embodiments may be considered to be distinct variations.

Example embodiments will now be described with reference to the accompanying drawings, which form a part of the present disclosure and which illustrate example embodiments which may be practiced. As used in the present disclosure and the appended claims, the terms “example embodiment”, “exemplary embodiment”, and “present embodiment” do not necessarily refer to a single embodiment, although they may, and various example embodiments may be readily combined and/or interchanged without departing from the scope or spirit of example embodiments. Furthermore, the terminology as used in the present disclosure and the appended claims is for the purpose of describing example embodiments only and is not intended to be limitations. In this respect, as used in the present disclosure and the appended claims, the term “in” may include “in” and “on”, and the terms “a”, “an”, and “the” may include singular and plural references. Furthermore, as used in the present disclosure and the appended claims, the term “by” may also mean “from”, depending on the context. Furthermore, as used in the present disclosure and the appended claims, the term “if” may also mean “when” or “upon”, depending on the context. Furthermore, as used in the present disclosure and the appended claims, the words “and/or” may refer to and encompass any and all possible combinations of one or more of the associated listed items.

### DETAILED DESCRIPTION

It is recognized in the present disclosure that conventional approaches to providing care for certain patients and the elderly, including those suffering from dementia and/or those who may aggravate, prolong, worsen, and/or create new health-related problems when they attempt to move, stand up, and/or walk, generally require one or more caregivers (e.g., nurses, family members, nannies, personal assistants, etc.) to be present and continuously monitor such patients.

With recent advances in technology, solutions have been introduced so as to enable caregivers to perform monitoring of patients without the need for caregivers to always be present, in line of sight, and/or within the confines of the patient's room. For example, closed-circuit television (CCTV) and other related technologies have enabled caregivers, such as nurses in hospitals, to continue visual and/or audio monitoring of patients from remote locations, such as from nursing stations.

While such solutions may allow caregivers to perform remote visual and/or audio monitoring of patients under their care, such solutions are generally only effective when caregivers either continuously monitor the patient via a CCTV television display or coincidentally glance over to the CCTV television display at a time when a needed situation arises. An example of a needed situation may include a situation where a patient suffering from dementia and/or other illnesses leaves his/her hospital bed, chair, or wheelchair and wanders away. Another example of a needed situation may include a situation where a post-surgery patient, who has been advised not to stand or walk, eventually leaves his/her hospital bed and takes a walk. Another example of a needed situation may include a non-medical-related situation, such as a situation where a person (e.g., a prisoner, detainee, or child) who has been requested, required, and/or restrained so as to sit or lie down on a particular chair, bench, bed, or spot on the floor, eventually stands up and/or walks away. As used in the present disclosure, a “user” may refer to any person, including a patient, elderly person, adult, teenager, child, toddler, infant, prisoner, detainee, suspect, employee, pilot, train conductor, and any person whose movement, non-movement, location, and/or identity may need to be detected, identified, verified, and/or monitored, and the like.

Present example embodiments relate generally to systems, devices, controllers, and methods for detecting, identifying, verifying, measuring, and/or monitoring, among other things, movement, non-movement, location, and/or identity of one or more users.

The System (e.g., System 100).

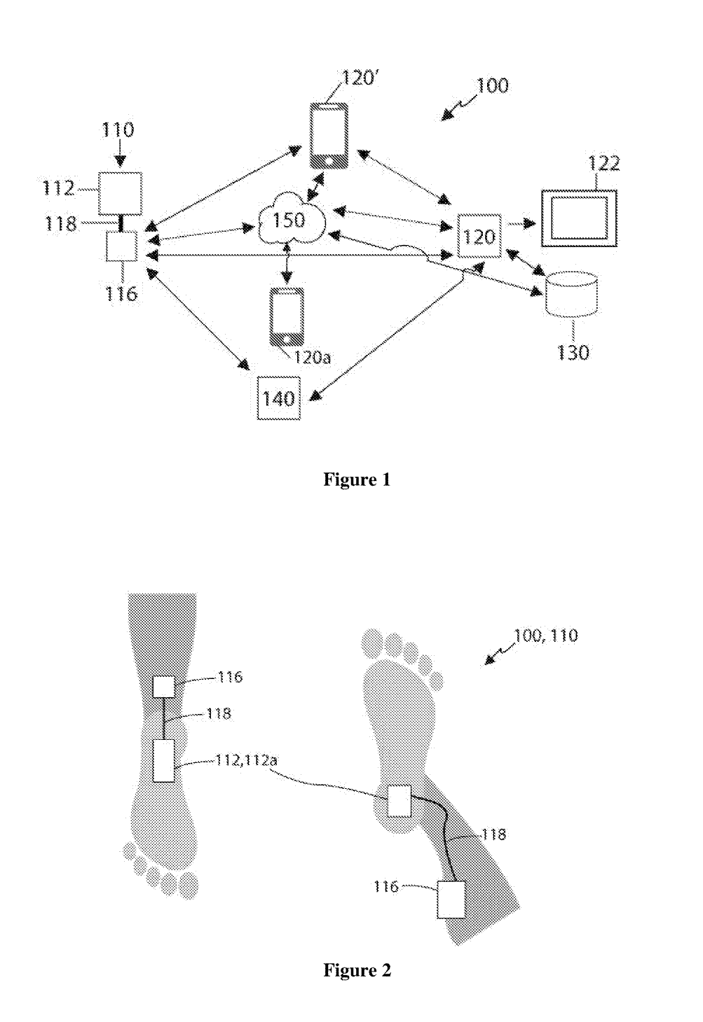

FIG. 1 illustrates an example embodiment of a system (e.g., system 100) for detecting, identifying, verifying, measuring, and/or monitoring (hereinafter “detecting”, “detect”, “monitoring”, or “monitor”) one or more movements, non-movements, locations, and/or identities (hereinafter “attribute” or “movement”) of one or more users. FIGS. 2-7 also illustrate example embodiments of such a system (e.g., system 100).

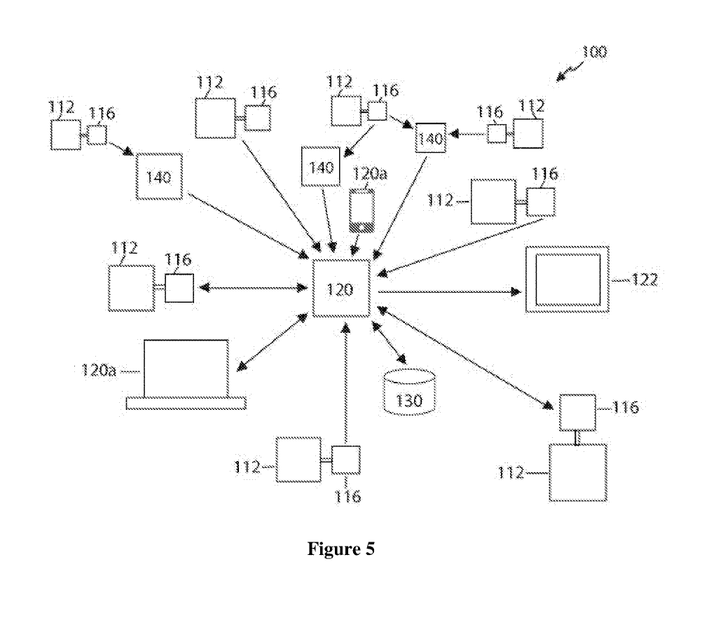

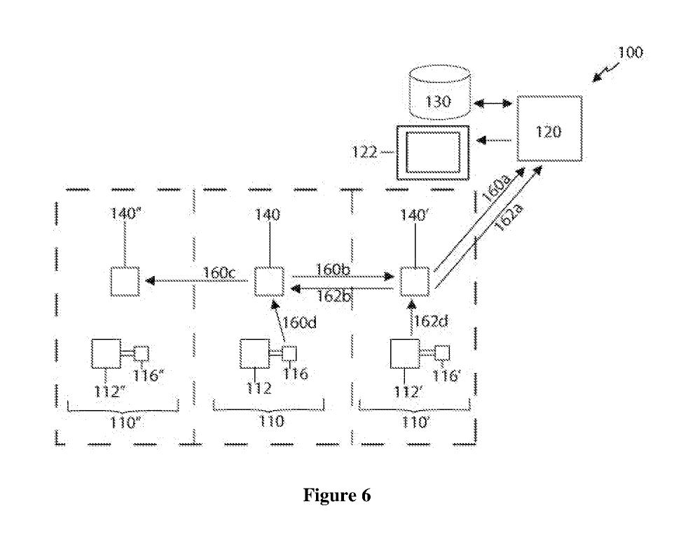

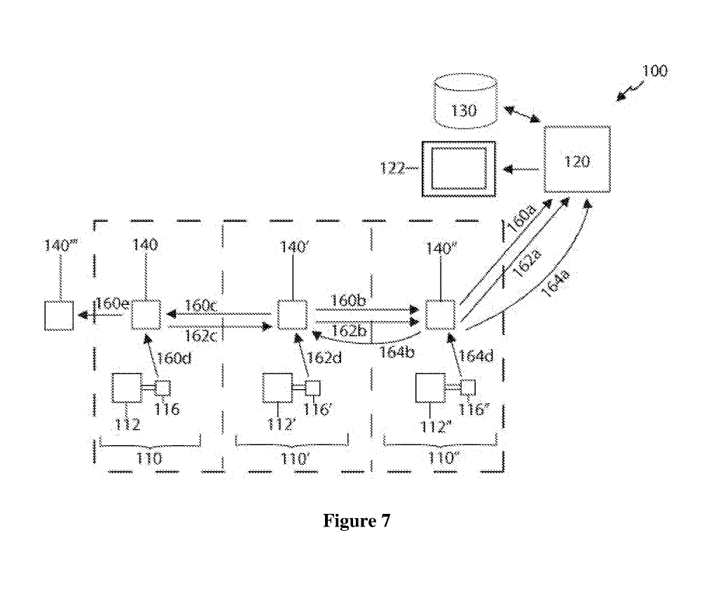

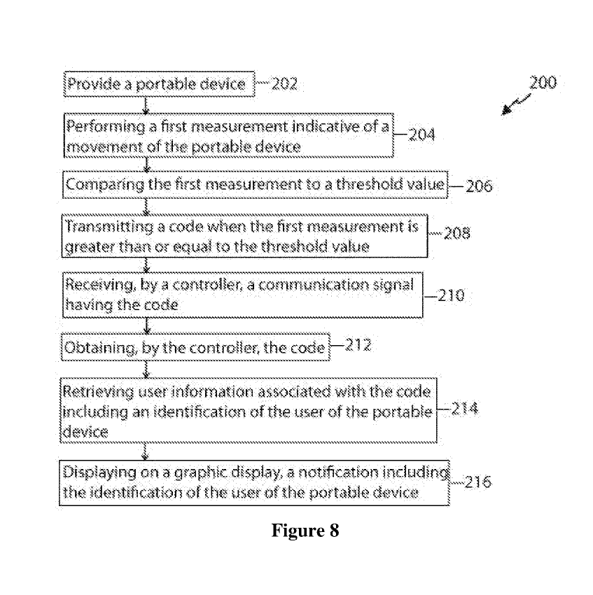

As illustrated in at least FIG. 1, the system (e.g., system 100) may comprise a portable device (e.g., portable device 110) and/or a controller (e.g., controller 120). Although FIG. 1 illustrates one portable device 110 and one controller 120, it is to be understood that example embodiments of the system 100 may comprise more than one portable device and/or more than one controller without departing from the teachings of the present disclosure. As a non-limiting example, the system (e.g., system 100) in FIG. 5 illustrates a plurality of portable devices 110. As another non-limiting example, the system (e.g., system 100) in FIG. 6 illustrates a plurality of portable devices 110, 110′, and 110″. As another non-limiting example, the system (e.g., system 100) in FIG. 7 illustrates a plurality of portable devices 110, 110′, 110″. In yet another non-limiting example, the system (e.g., system 100) in FIG. 1 illustrates more than one controller 120 and 120′.

The system (e.g., system 100) may also comprise one or more databases (e.g., database 130). Each database (e.g., database 130) may be in communication with one or more elements of the system (e.g., system 100), such as controller (e.g., controller 120) and/or network (e.g., network 150). Each database (e.g., database 130) may be configurable to store, among other things, information pertaining to a portable device (e.g., portable device 110), user of the portable device (e.g., portable device 110), repeater (e.g., repeater 140), caregiver device (e.g., caregiver device 120a), controllers (e.g., controller 120), network (e.g., network 150), and/or location(s) of the portable device (e.g., portable device 110), user of the portable device (e.g., portable device 110), repeater (e.g., repeater 140), caregiver device (e.g., caregiver device 120a), controllers (e.g., controller 120), and/or elements of the network (e.g., network 150). Although the figures illustrate one database 130, it is to be understood that example embodiments of the system 100 may comprise more than one database without departing from the teachings of the present disclosure.

One or more graphical displays (e.g., graphical display 122) may also be included in the system (e.g., system 100). Each graphical display (e.g., graphical display 122) may be in communication with one or more elements of the system (e.g., system 100), such as the controller (e.g., controller 120 and/or controller 120′), portable device (e.g., portable device 110), database (e.g., database 130), and/or repeater (e.g., repeater 140). Each graphical display (e.g., graphical display 122) may be configurable to receive, from the controller (e.g., controller 120) and/or database (e.g., database 130), information (e.g., a notification message) and display the information (e.g., the notification message) on the graphical display (e.g., graphical display 122). As will be further described in the present disclosure, the notification message may include information pertaining to a portable device (e.g., portable device 110), a user of the portable device (e.g., portable device 110), a location of a portable device (e.g., portable device 110) and/or user of the portable device (e.g., portable device 110), and/or an indication of a detection of movement of the portable device (e.g., portable device 110) and/or use of the portable device (e.g., portable device 110). Although the figures illustrate one graphical display 122, it is to be understood that example embodiments of the system 100 may comprise more than one graphical display without departing from the teachings of the present disclosure.

The system (e.g., system 100) may also comprise one or more repeaters (e.g., repeater 140). Each repeater (e.g., repeater 140) may be configurable to communicate with one or more elements of the system (e.g., system 100). Although FIG. 1 illustrates one repeater 140, it is to be understood that example embodiments of the system 100 may comprise more than one repeater without departing from the teachings of the present disclosure. As a non-limiting example, the system (e.g., system 100) in FIG. 5 illustrates a plurality of repeaters 140. As another non-limiting example, the system (e.g., system 100) in FIG. 6 illustrates a plurality of repeaters 140, 140′, and 140″. In yet another non-limiting example, the system (e.g., system 100) in FIG. 7 illustrates a plurality of repeaters 140, 140′, 140″, and 140′″.

One or more caregiver devices (e.g., caregiver device 120a in FIGS. 1 and 5) may also be included in the system (e.g., system 100). Although FIG. 1 illustrates one caregiver device 120a, it is to be understood that example embodiments of the system 100 may comprise more than one caregiver device without departing from the teachings of the present disclosure. As a non-limiting example, the system (e.g., system 100) in FIG. 5 illustrates a plurality of caregiver devices 120a.

The system (e.g., system 100) may also comprise one or more networks (e.g., network 150). Although FIG. 1 illustrates one network 150, it is to be understood that example embodiments of the system 100 may comprise more than one network without departing from the teachings of the present disclosure.

These and other example embodiments will now be described with reference to the accompanying drawings.

The Portable Device (e.g., Portable Device 110).

The system (e.g., system 100) may comprise one or more portable devices (e.g., portable device 110) in example embodiments. Each portable device (e.g., portable device 110) may be configurable to communicate with one or more elements of the system (e.g., system 100). As a non-limiting example, each portable device (e.g., portable device 110) may be configurable to communicate, including receive and transmit information, with other portable devices (e.g., portable device 110, such as when some or all portions of the system 100 are configured as a mesh network, or the like), one or more repeaters (e.g., repeater 140, such as when some or all portions of the system 100 are configured as a mesh network, or the like), one or more portable devices (e.g., portable device 110), one or more controllers (e.g., controller 120), one or more databases (e.g., database 130), one or more networks (e.g., network 150), and/or one or more caregiver devices (e.g., caregiver devices 120a). Such communication between each portable device (e.g., portable device 110) and one or more of the elements of the system (e.g., system 100) described above and in the present disclosure may be in one or more of a variety of forms, including, but not limited to, omni-directional broadcasting, directed or directional broadcasting, peer-to-peer communications, mesh-type communications, connected communications, non-connected communications, full-duplex-type communications, half duplex-type communications, etc. As a non-limiting example, a portable device (e.g., portable device 110) may send a communication signal to a controller (e.g., controller 120) via one or more other portable devices (e.g., portable device 110) and/or one or more repeaters (e.g., repeater 140), all of which may be configured or configurable to be in a broadcast-type, non-connected, and/or half-duplex mesh network.

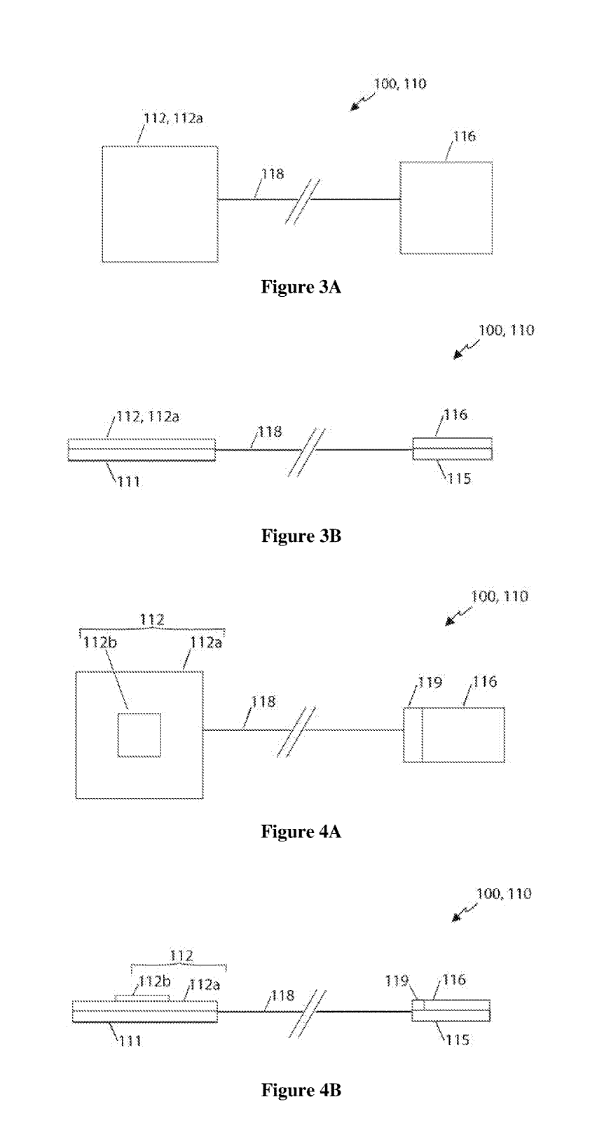

As illustrated in at least the perspective view of FIG. 2 and top view of FIG. 3A, an example embodiment of the portable device (e.g., portable device 110) may include a sensor assembly (e.g., sensor assembly 112) and a processor (e.g., processor 116). The sensor assembly (e.g., sensor assembly 112) and the processor (e.g., processor 116) may be configurable to communicate with one another wirelessly (not shown) and/or via one or more wires (e.g., wire 118). Although the figures illustrate the sensor assembly (e.g., sensor assembly 112) and the processor (e.g., processor 116) to be physically separate elements, it is to be understood that example embodiments of the portable device (e.g., portable device 110) may provide for the sensor assembly (e.g., sensor assembly 112) and the processor (e.g., processor 116) to be attachable, securable, connectable, and/or adjacent to one another, and/or formed as a unitary article, without departing from the teachings of the present disclosure. Furthermore, although the figures illustrate each of the sensor assembly (e.g., sensor assembly 112) and the processor (e.g., processor 116) to be single or unitary elements, it is to be understood that example embodiments may provide for one or more of the sensor assembly (e.g., sensor assembly 112) and the processor (e.g., processor 116) to be physically separate elements.

The portable device (e.g., portable device 110) may also comprise an energy source (e.g., energy source 119), as illustrated in the top view of FIG. 4A and cross-sectional view of FIG. 4B. Although FIGS. 4A and 4B illustrate one energy source 119, it is to be understood that example embodiments of the portable device (e.g., portable device 110) may include more than one energy source without departing from the teachings of the present disclosure. As a non-limiting example, the sensor assembly (e.g., sensor assembly 112) and the processor (e.g., processor 116) may have one or more shared energy sources, as shown in FIGS. 4A and 4B. As another non-limiting example, the sensor assembly (e.g., sensor assembly 112) and the processor (e.g., processor 116) may each have its own one or more energy sources (not shown).

In an example embodiment, the portable device (e.g., portable device 110) may also include a location-based assembly (not shown). The location-based assembly may be configurable to determine a location of the portable device (e.g., portable device 110). As a non-limiting example, the location-based assembly may include a global positioning system (GPS), or the like, configurable to determine a location of the portable device (e.g., portable device 110). As another non-limiting example, the location-based assembly may include functionality to perform, or assist in performing, a triangulation of signals between one or more elements of the system (e.g., system 100), such as one or more repeaters (e.g., repeater 140), one or more other portable devices (e.g., portable device 110), one or more caregiver devices (e.g., caregiver device 120a), and/or one or more elements of or in communication with the network (e.g., network 150) and/or the system (e.g., system 100). In an example embodiment, the code transmitted by the processor (e.g., processor 116) may include, among other things, the location of the portable device (e.g., portable device 110), as determined by the location-based assembly. Furthermore, as described in the present disclosure, a notification message displayed, on the graphical display, may include the location of the portable device (e.g., portable device 110), as determined by the location-based assembly.

The portable device (e.g., portable device 110) may also include one or more attachable portions (e.g., attachable portions 111, 115), as illustrated in the cross-sectional views of FIG. 3B and FIG. 4B, which may be any adhesive, tape, tie, medical-grade tape, Velcro-based strap, magnetic-based strap, etc. for use in securing, attaching, coupling, holding, etc., one or more elements of the portable device (e.g., portable device 110), including the sensor assembly (e.g., sensor assembly 112) and/or the processor (e.g., processor 116) to a portion of the user, user's article of clothing, etc.

(i) Energy Source (e.g., Energy Source 119).

In an example embodiment, the portable device (e.g., portable device 110) may include one or more energy sources (e.g., energy source 119). The one or more energy sources (e.g., energy source 119) may be configurable to continuously, intermittently, and/or periodically provide or supply current (or power, voltage, or energy) to the sensor assembly (e.g., sensor assembly 112) and/or the processor (e.g., processor 116) in example embodiments. In addition to or alternatively, the one or more energy sources (e.g., energy source 119) may be configurable to provide or supply current (or power, voltage, or energy) to the sensor assembly (e.g., sensor assembly 112) and/or the processor (e.g., processor 116) on an on-demand basis, after being triggered, upon one or more events occurring, and/or upon satisfying one or more conditions. In addition to or alternatively, the one or more energy sources (e.g., energy source 119) may be configurable to provide or supply current (or power, voltage, or energy) to one of the sensor assembly (e.g., sensor assembly 112) and the processor (e.g., processor 116), and said one of the sensor assembly (e.g., sensor assembly 112) and the processor (e.g., processor 116) may in turn be configurable to provide or supply current (or power, voltage, or energy) to the other.

In an example embodiment, the sensor assembly (e.g., sensor assembly 112) may be configurable to be in an off state, which is a state and/or operating mode in respect of which the sensor assembly (e.g., sensor assembly 112) does not receive a current (or power, voltage, or energy) from the energy source (e.g., energy source 119). The sensor assembly (e.g., sensor assembly 112) may also be configurable to be in an on state, which is a state and/or operating mode in respect of which the sensor assembly (e.g., sensor assembly 112) receives a current (or power, voltage, or energy) from the energy source (e.g., energy source 119). The sensor assembly (e.g., sensor assembly 112) may also be transitionable between the off state and the on state in example embodiments. The sensor assembly (e.g., sensor assembly 112) may also be configurable to be in states other than the off state and the on state without departing from the teachings of the present disclosure. In such embodiments, the sensor assembly (e.g., sensor assembly 112) may also be transitional between such other states and the off state and on state.

In addition to or in replacement, the processor (e.g., processor 116) may be configurable to be in an off state, which is a state and/or operating mode in respect of which the processor (e.g., processor 116) does not receive a current (or power, voltage, or energy) from the energy source (e.g., energy source 119). The processor (e.g., processor 116) may also be configurable to be in an on state, which is a state and/or operating mode in respect of which the processor (e.g., processor 116) receives a current (or power, voltage, or energy) from the energy source (e.g., energy source 119). The processor (e.g., processor 116) may also be transitionable between the off state and the on state in example embodiments. The processor (e.g., processor 116) may also be configurable to be in states other than the off state and the on state without departing from the teachings of the present disclosure. In such embodiments, the processor (e.g., processor 116) may also be transitional between such other states and the off state and on state.

The energy source (e.g., energy source 119) may be any energy source, or the like, including, but not limited to, a standard battery, rechargeable lithium ion battery, wirelessly rechargeable battery, bio-based battery (or bio-battery), solar-based battery, or the like.

(ii) The Sensor Assembly (e.g., Sensor Assembly 112).

In an example embodiment, the portable device (e.g., portable device 110) may include one or more sensor assemblies (e.g., sensor assembly 112). The sensor assembly (e.g., sensor assembly 112) may include a sensor (e.g., sensor 112a), as illustrated in the cross-sectional view of FIG. 3B and various views of FIGS. 2, 3A, and 4A-B. Although the figures illustrate one sensor 112a, it is to be understood that example embodiments of the sensor assembly (e.g., sensor assembly 112) may include more than one sensor without departing from the teachings of the present disclosure. Furthermore, although the figures may illustrate the sensor (e.g., sensor 112a) to be a single or unitary element, it is to be understood that example embodiments may provide for the sensor (e.g., sensor 112a) to be physically separate elements.

The sensor (e.g., sensor 112a) may be any sensor, detector, switch, accelerometer, gyroscope, RF-based device, magnetic-based device, chemical-based device, temperature-based device, pressure-based device, location-based device, bio-based device, orientation-detecting sensor, moisture-detecting sensor, photo-detecting sensor, light-detecting sensor, brightness-detecting sensor, ultraviolet-detecting sensor, infrared-detecting sensor, radiation-detecting sensor, or the like, configurable to perform, among other things, monitoring of a predetermined action (or inaction). The predetermined action may be any action (or inaction) indicative of a movement (or non-movement) of the portable device (e.g., portable device 110), one or more elements of the portable device (e.g., portable device 110), and/or user of the portable device (e.g., portable device 110). As a non-limiting example, the predetermined action may be a predetermined action performed on at least one portion of the sensor assembly (e.g., sensor assembly 112). As another non-limiting example, the predetermined action may be an application of a force or pressure (hereinafter “applied force”) on at least one portion of the sensor assembly (e.g., sensor assembly 112). As another non-limiting example, the predetermined action may be an applied force on the sensor (e.g., sensor 112a), a switch (e.g., switch 112b), and/or other portion of the sensor assembly (e.g., sensor assembly 112), which may be detectable when the sensor (e.g., sensor 112) includes, for example, a force sensor or force sensing resistor. As another non-limiting example, the predetermined action may be a singular movement or sequence of movements of the portable device (e.g., portable device 110), one or more elements of the portable device (e.g., portable device 110), and/or user of the portable device (e.g., portable device 110), which may be detectable when the sensor (e.g., sensor 112a) includes, for example, an accelerometer and/or gyroscope.

The sensor (e.g., sensor 112a) may be a force sensor, force sensing resistor, pressure sensor, temperature sensor, acceleration sensor, accelerometer, gyroscope, displacement sensor, RF-based sensor, magnetic-based sensor, chemical-based sensor, temperature-based sensor, pressure-based sensor, location-based sensor, bio-based sensor, orientation-detecting sensor, moisture-detecting sensor, photo-detecting sensor, light-detecting sensor, brightness-detecting sensor, ultraviolet-detecting sensor, infrared-detecting sensor, radiation-detecting sensor, or the like, in example embodiments.

In example embodiments wherein the sensor (e.g., sensor 112a) is a force sensor 112a, or the like, the force sensor 112a may be configurable to receive an applied force (or impact of force) and perform a predetermined action (or refrain from performing an action) in response to the applied force. In an example embodiment, the force sensor 112a may be configurable to receive an applied force and, based on the magnitude (and/or direction and/or duration) of the applied force, selectively send (or not send) a command to the processor (e.g., processor 116) and/or other element(s) of the portable device (such as the energy source 119) and/or system (e.g., system 100). The command may be any signal, electric current, voltage potential, resistance, opening or closing of a switch, or the like.

As a non-limiting example, the force sensor 112a may be configurable to receive an application of force, detect and/or measure the applied force (quantitatively and/or qualitatively), and provide, send, or make available the detected and/or measured applied force to the processor (e.g., processor 116) (as either a quantitative amount and/or a qualitative amount), such as when the applied force is greater than or equal to a cutoff value. The cutoff value may be any value, such as a force value based on a weight of the user of the portable device (e.g., portable device 110). As a non-limiting example, the cutoff value may be a force value equivalent to 10-90% of the weight of the user, or more preferably, 30-70% of the weight of the user, or even more preferably, 40-60% of the weight of the user. The cutoff value may also be a predetermined value based on one or more criteria, including, but not limited to, age, weight, height, etc. of the user of the portable device and/or average user without departing from the teachings of the present disclosure. As will be further explained below, upon receiving the detected and/or measured applied force from the force sensor 112a, the processor (e.g., processor 116) may be configurable to perform, among other things, a comparison of the detected and/or measured applied force to a threshold value. The processor (e.g., processor 116) may be further configurable to transmit a code, such as via a communication signal, or the like, when the processor determines, based on the comparison, that the detected and/or measured applied force is greater than or equal to the threshold value. Such transmission of the code, such as via a communication signal, may be a transmission to one or more elements of the system (e.g., system 100) including, but not limited to, the controller (e.g., controller 120, 120′), caregiver device (e.g., caregiver device 120a), network (e.g., network 150), repeater (e.g., repeater 140), and/or another portable device (e.g., portable device 110).

As another non-limiting example, the force sensor 112a may be configurable to perform one or more of the following: receive an applied force; detect and/or measure the applied force (quantitatively and/or qualitatively); send a command to the energy source (e.g., energy source 119) to selectively allow the energy source 119 to provide or supply current (or power, voltage, or energy) to the processor (e.g., processor 116) (i.e., transition the processor from the off state to the on state, such as when the processor is in a normally off state) when the applied force is greater than or equal to the cutoff value; and provide, send, or make available the detected and/or measured applied force to the processor (e.g., processor 116). As introduced above and will be further explained below, upon receiving the detected and/or measured applied force from the force sensor 112a, the processor (e.g., processor 116) may be configurable to perform, among other things, a comparison of the detected and/or measured applied force to the threshold value. The processor (e.g., processor 116) may be further configurable to transmit a code, such as via a communication signal, when the processor determines, based on the compare, that the detected and/or measured applied force is greater than or equal to the threshold value. Such transmission of the code may be to one or more elements of the system (e.g., system 100) including, but not limited to, the controller (e.g., controller 120, 120′), caregiver device (e.g., caregiver device 120a), network (e.g., network 150), repeater (e.g., repeater 140), and/or another portable device (e.g., portable device 110).

In an example embodiment, the sensor assembly (e.g., sensor assembly 112) may also comprise a switch (e.g., switch 112b), or the like, as illustrated in FIGS. 4A-B. Although the figures illustrate one switch 112b, it is to be understood that example embodiments of the sensor assembly (e.g., sensor assembly 112) may include more than one switch without departing from the teachings of the present disclosure. Furthermore, although the cross-sectional view of FIG. 4B illustrates the switch (e.g., switch 112b) as protruding above or higher than the top surface of the sensor (e.g., sensor 112a), it is to be understood that the switch (e.g., switch 112b) may be provided in such a way that a top surface of the switch (e.g., switch 112b) is at about the same height as or flush with the top surface of the sensor (e.g., sensor 112a). Furthermore, although the top view of FIG. 4A illustrates the switch (e.g., switch 112b) as being centered with respect to the sensor (e.g., sensor 112a), it is to be understood that the switch (e.g., switch 112b) may be provided at any location with respect to the sensor (e.g., sensor 112a), including near to or adjacent to one of the sides of the switch (e.g., switch 112b) or entirely separated from the switch (e.g., switch 112b).

The switch (e.g., switch 112b) may be any sensor, detector, switch, accelerometer, gyroscope, RF-based device, magnetic-based device, chemical-based device, temperature-based device, pressure-based device, location-based device, bio-based device, orientation-detecting sensor, moisture-detecting sensor, photo-detecting sensor, light-detecting sensor, brightness-detecting sensor, ultraviolet-detecting sensor, infrared-detecting sensor, radiation-detecting sensor, or the like, configurable to perform, among other things, transitioning of itself and/or one or more elements of the portable device (e.g., portable device 110) between an on state and an off state upon receiving a certain action (e.g., certain applied force).

As a non-limiting example, the switch (e.g., switch 112b) may be a contact switch 112b, or the like, configurable to transition one or more elements of the portable device (e.g., portable device 110) between an off state and an on state. As a more specific non-limiting example, in an embodiment where the sensor (e.g., sensor 112a) is in a normally off state (or normally on state, as applicable), the contact switch 112b may, upon receiving a certain magnitude of force (i.e., contact or impact) applied to it, transition the sensor (e.g., sensor 112a) from its normally off state to an on state (or vice versa, as applicable). The said magnitude of applied force on the contact switch 112b may be the cutoff value, as described above and in the present disclosure, or any other value. As another non-limiting example, in an embodiment where the processor (e.g., processor 116) is in a normally off state (or normally on state, as applicable), the contact switch 112b may, upon receiving a certain magnitude of force (i.e., contact or impact) applied to it, transition the processor (e.g., processor 116) from its normally off state to an on state (or vice versa, as applicable). The said magnitude of applied force on the contact switch 112b may be the cutoff value, as described above and in the present disclosure, or any other value. In yet another non-limiting example, in an embodiment where the sensor (e.g., sensor 112a) and processor (e.g., processor 116) are each in a normally off state (or normally on state, as applicable), the contact switch 112b may, upon receiving a certain magnitude of force (i.e., contact or impact) applied to it, transition the sensor (e.g., sensor 112a) and processor (e.g., processor 116), either simultaneously or sequentially, from their normally off state to an on state (or vice versa, as applicable). The said magnitude of applied force on the contact switch 112b may be the cutoff value, as described above and in the present disclosure, or any other value.

As described above and in the present disclosure, the sensor (e.g., sensor 112a) may be transitionable between an off state and an on state.

In an example embodiment, the off state (or normally off state) may be a state and/or operating mode in respect of which the sensor (e.g., sensor 112a) does not receive a current (or power, voltage, or energy) from the energy source (e.g., energy source 119). In example embodiments, the sensor (e.g., sensor 112a) may not perform some or all of the operations of the sensor (e.g., sensor 112a), such as the measuring of the applied force, sending of a command to the energy source (e.g., energy source 119) to transition the processor to the on state (or off state, as applicable), and/or providing of the measured applied force to the processor, while the sensor (e.g., sensor 112a) is in the off state (or normally off state). In a more specific non-limiting example, the sensor (e.g., sensor 112a) may not perform any measuring of the applied force, sending of a command to the energy source (e.g., energy source 119) to transition the processor to the on state (or off state, as applicable), and providing of the measured applied force to the processor while the sensor (e.g., sensor 112a) is in the off state (or normally off state).

In an example embodiment, the on state (or normally on state) may be a state and/or operating mode in respect of which the sensor (e.g., sensor 112a) receives a current (or power, voltage, or energy) from the energy source (e.g., energy source 119). In example embodiments, the sensor (e.g., sensor 112a) may perform some or all of the operations of the sensor (e.g., sensor 112a), such as the measuring of the applied force, sending of a command to the energy source (e.g., energy source 119) to transition the processor to the on state (or off state, as applicable), and/or providing of the measured applied force to the processor, while the sensor (e.g., sensor 112a) is in the on state (or normally on state). In a more specific non-limiting example, the sensor (e.g., sensor 112a) may perform measuring of the applied force, sending of a command to the energy source (e.g., energy source 119) to transition the processor to the on state (or off state, as applicable), and providing of the measured applied force to the processor while the sensor (e.g., sensor 112a) is in the on state (or normally on state).

The sensor assembly (e.g., sensor assembly 112) may also be configurable to be in states other than the off state and the on state (and the normally off state and the normally on state), such as a sleep state, transitional state, semi-operational state, or the like, without departing from the teachings of the present disclosure. In such embodiments, the sensor assembly (e.g., sensor assembly 112) may also be transitional between such states and the off state and on state (and normally off state and normally on state).

(iii) The Processor (e.g., Processor 116).

In an example embodiment, the portable device (e.g., portable device 110) may also comprise one or more processors (e.g., processor 116). Although the figures illustrate one processor 116, it is to be understood that example embodiments of the portable device (e.g., portable device 110) may include more than one processor without departing from the teachings of the present disclosure. Furthermore, although the figures may illustrate the processor (e.g., processor 116) to be a single or unitary element, it is to be understood that example embodiments may provide for the processor (e.g., processor 116) to be physically separate elements.

The processor (e.g., processor 116) may be any processor, microprocessor, controller, microcontroller, microchip, mobile device, semiconductor device, or the like, configurable to perform a processing of information and/or other predetermined actions. The processor (e.g., processor 116) described herein may also include and/or be a part of a wearable computing device. The processor (e.g., processor 116) may also include or be a part of a virtual machine, computer, node, instance, host, or machine in a networked computing environment. As used in the present disclosure, such a network, and/or cloud, may be a collection of devices connected by communication channels that facilitate communications between devices and allow for devices to share resources. Such resources may encompass any types of resources for running instances including hardware (such as servers, clients, mainframe computers, networks, network storage, data sources, memory, central processing unit time, scientific instruments, and other computing devices), as well as software, software licenses, available network services, and other non-hardware resources, or a combination thereof. A network or cloud may include, but is not limited to, computing grid systems, peer to peer systems, mesh-type systems, distributed computing environments, cloud computing environment, etc. Such network or cloud may include hardware and software infrastructures configured to form a virtual organization comprised of multiple resources which may be in geographically disperse locations. Network may also refer to a communication medium between processes on the same device. Also as referred to herein, a network element, node, or server may be a device deployed to execute a program operating as a socket listener and may include software instances.

In an example embodiment, the information processed or processable by the processor (e.g., processor 116) may be any information, including information to detect, measure, and/or confirm a certain movement (or non-movement) of the portable device (e.g., portable device 110), one or more elements of the portable device, and/or user of the portable device. As a non-limiting example, the information processed or processable by the processor (e.g., processor 116) may be information pertaining to an action (or non-action) performed on at least one portion of the sensor assembly (e.g., sensor assembly 112). As another non-limiting example, the information processed or processable by the processor (e.g., processor 116) may be information pertaining to an applied force on at least one portion of the sensor assembly (e.g., sensor assembly 112). As another non-limiting example, the information processed or processable by the processor (e.g., processor 116) may be information pertaining to an applied force on the sensor (e.g., sensor 112a), switch (e.g., switch 112b), and/or other portion of the sensor assembly (e.g., sensor assembly 112), which may be detectable when the sensor (e.g., sensor 112) includes, for example, a force sensor or force sensing resistor. As another non-limiting example, the information processed or processable by the processor (e.g., processor 116) may be information pertaining to a singular movement or sequence of movements of the portable device (e.g., portable device 110), one or more elements of the portable device (e.g., portable device 110), and/or user of the portable device (e.g., portable device 110), which may be detectable when the sensor (e.g., sensor 112a) includes, for example, an accelerometer and/or gyroscope.

As introduced above, in example embodiments wherein the sensor (e.g., sensor 112a) is a force sensor 112a, or the like, the force sensor 112a may be configurable to receive an application of force, detect and/or measure the applied force (quantitatively and/or qualitatively), and provide, send, or make available the detected and/or measured applied force to the processor (e.g., processor 116) (as either a quantitative amount and/or a qualitative amount) when the applied force is greater than or equal to the cutoff value.

Upon receiving the detected and/or measured applied force from the force sensor 112a, the processor (e.g., processor 116) may be configurable to process the detected and/or measured applied force. As a non-limiting example, the processor (e.g., processor 116) may be configurable to perform a comparison of the detected and/or measured applied force to a threshold value or any other value. The threshold value may be greater than or equal to the cutoff value in example embodiments. The threshold value may be any value, such as a force value based on a weight of the user of the portable device (e.g., portable device 110). As a non-limiting example, the threshold value may be equivalent to 40-100% of the weight of the user, or more preferably, 60-100% of the weight of the user, or even more preferably, 80-100% of the weight of the user. The threshold value may also be a predetermined value based on one or more criteria, including, but not limited to, age, weight, height, etc. of the user of the portable device and/or average user without departing from the teachings of the present disclosure.

In an example embodiment, the processor (e.g., processor 116) may perform the processing described above and in the present disclosure so as to make a determination as to whether or not the user of the portable device (e.g., portable device 110) has performed (or not performed) one or more of the following actions: move (e.g., sensor assembly may be placed on one or some parts of the user's body), sit up (e.g., sensor assembly may be placed on a buttock and/or back side of the user's body), stand up (e.g., sensor assembly may be placed on a bottom portion of one or both feet of the user, such as a heel and/or a ball of a foot), fall down (e.g., sensor assembly may be placed on one or both knees of the user, one or both palms of the user, and/or on a buttock and/or back side of the user's body), walk (e.g., sensor assembly may be placed on a bottom of one or both feet of the user), jog (e.g., sensor assembly may be placed on a bottom of one or both feet of the user), and/or run (e.g., sensor assembly may be placed on a bottom of one or both feet of the user). Other user actions are also contemplated without departing from the teachings of the present disclosure.

When the processor (e.g., processor 116) determines, based on the processing (e.g., comparison of the measured applied force to a threshold value), that the measured applied force is greater than or equal to the threshold value, the processor (e.g., processor 116) may be further configurable to transmit a code. The code may be transmitted by the processor (e.g., processor 116) in any form or manner, such as via a communication signal, or the like. Such transmission of the code may be a transmission to one or more elements of the system (e.g., system 100) including, but not limited to, the controller (e.g., controller 120, 120′), caregiver device (e.g., caregiver device 120a), network (e.g., network 150), repeater (e.g., repeater 140), and/or another portable device (e.g., portable device 110). In a non-limiting example, the processor (e.g., processor 116) may be or include a Bluetooth low energy (LE)-enabled chip, or the like, configurable to communicate the code via Bluetooth, Wi-Fi, the internet, radio frequency (RF), infrared (IF), or the like. In example embodiments, communications between the processor (e.g., processor 116) and one or more of the elements of the system (e.g., system 100) described above and in the present disclosure may be in one or more of a variety of forms, including, but not limited to, omni-directional broadcasting, directed or directional broadcasting, peer-to-peer communications, mesh-type communications, connected communications, non-connected communications, full-duplex-type communications, half duplex-type communications, etc. As a non-limiting example, the processor (e.g., processor 116) may send the code or communication signal having the code to a controller (e.g., controller 120) via one or more other processors (e.g., processor 116) of other portable devices and/or one or more repeaters (e.g., repeater 140), all of which may be configured or configurable to be in a broadcast-type, non-connected, and/or half-duplex mesh network.

As described above and in the present disclosure, the processor (e.g., processor 116) may be transitionable between an off state and an on state.

In an example embodiment, the off state (or normally off state) may be a state and/or operating mode in respect of which the processor (e.g., processor 116) does not receive a current (or power, voltage, or energy) from the energy source (e.g., energy source 119). In example embodiments, the processor (e.g., processor 116) may not perform some or all of the operations of the processor (e.g., processor 116), such as the receiving of information, processing of information, and/or transmitting of information, while the processor (e.g., processor 116) is in the off state (or normally off state). In a more specific non-limiting example, the processor (e.g., processor 116) may not perform any receiving of information (e.g., detected and/or measured applied force), processing of information (e.g., comparison of the measured applied force to a threshold value), and transmitting of information (e.g., transmission of the code) while the processor (e.g., processor 116) is in the off state (or normally off state).

In an example embodiment, the on state (or normally on state) may be a state and/or operating mode in respect of which the processor (e.g., processor 116) receives a current (or power, voltage, or energy) from the energy source (e.g., energy source 119). In example embodiments, the processor (e.g., processor 116) may perform some or all of the operations of the processor (e.g., processor 116), such as the receiving of information, processing of information, and/or transmitting of information, while the processor (e.g., processor 116) is in the on state (or normally on state). In a more specific non-limiting example, the processor (e.g., processor 116) may perform the receiving of information (e.g., detected and/or measured applied force), processing of information (e.g., comparison of the measured applied force to a threshold value), and transmitting of information (e.g., transmission of the code) while the processor (e.g., processor 116) is in the on state (or normally on state).

The processor (e.g., processor 116) may also be configurable to be in states other than the off state and the on state (and normally off state and normally on state), such as a sleep state, transitional state, semi-operational state, or the like, without departing from the teachings of the present disclosure. In such embodiments, the processor (e.g., processor 116) may also be transitional between such states and the off state and on state (and normally off state and normally on state).

The Controller (e.g., Controller 120).

In an example embodiment, the system (e.g., system 100) may comprise the controller (e.g., controller 120). Although the figures illustrate one controller 120, it is to be understood that example embodiments of the system (e.g., system 100) may include more than one controller without departing from the teachings of the present disclosure.

The controller (e.g., controller 120) may be any processor, microprocessor, controller, microcontroller, microchip, mobile device, semiconductor device, or the like, configurable to perform a processing of information and/or other predetermined actions. The controller (e.g., controller 120) described herein may be directed to and/or include desktop computers, all-in-one computers, laptops, notebooks, ultrabooks, readers, televisions/monitors, appliances, tablets, phablets, mobile phones, PDAs, media players, a plurality of computing devices interacting together in part or in whole, and other specialized computing devices and industry-specific computing devices. The controller (e.g., controller 120) described herein may also be directed to wearable computing devices, including glasses (such as Google Glasses), watches (such as the Apple Watch), etc. In such applications, it is to be understood that the controller (e.g., controller 120) may also process gestures and/or voice of the user, caregiver, and/or other persons using the controller (e.g., controller 120). The controller (e.g., controller 120) may also may be a virtual machine, computer, node, instance, host, or machine in a networked computing environment. Such networked environment, and/or cloud, may be a collection of machines connected by communication channels that facilitate communications between machines and allow for machines to share resources. Such resources may encompass any types of resources for running instances including hardware (such as servers, clients, mainframe computers, networks, network storage, data sources, memory, central processing unit time, scientific instruments, and other computing devices), as well as software, software licenses, available network services, and other non-hardware resources, or a combination thereof. A network or cloud may include, but is not limited to, computing grid systems, peer to peer systems, mesh-type systems, distributed computing environments, cloud computing environment, etc. Such network, and/or cloud, may include hardware and software infrastructures configured to form a virtual organization comprised of multiple resources which may be in geographically disperse locations. Network may also refer to a communication medium between processes on the same machine. Also as referred to herein, a network element, node, or server may be a machine deployed to execute a program operating as a socket listener and may include software instances.

The controller (e.g., controller 120) may be in communication with one or more portable devices (e.g., portable device 110). For example, the controller (e.g., controller 120) may be configurable to receive communication signals, or the like, including a communication signal having the code transmitted from one or more portable devices (e.g., portable device 110). In this regard, the controller (e.g., controller 120) may also be configurable to obtain, from such each communication signal transmitted from a portable device (e.g., portable device 110), the code.

The controller (e.g., controller 120) may also be in communication with one or more databases (e.g., database 130), each of which may store information, including one or more of the codes and user information associated with one or more of the codes. For example, upon obtaining a code from a communication signal transmitted by a portable device (e.g., portable device 110), the controller (e.g., controller 120) may be configurable to retrieve, from one or more databases (e.g., database 130), one or more pieces of user information associated with the obtained code. Such retrieving may be performed based on the obtained code, and such retrieved user information may include, among other things, an identification of the user of the portable device (e.g., portable device 110). The retrieved user information may also include an identification of a particular one or more caregivers assigned to provide care for the user of the portable device (e.g., portable device 110) based on one or more criteria. For example, the retrieved user information may include an identification of a particular caregiver assigned to provide care for the user of the portable device (e.g., portable device 110) for a specific time or period (e.g., the time the controller receives the communication signal, the time the processor sends the communication signal, etc.), and may also include an identification of another caregiver assigned to be a backup caregiver.

The controller (e.g., controller 120) may also be in communication with one or more repeaters (e.g., repeater 140). Such repeaters (e.g., repeater 140) may be configurable to perform one or more operations. For example, each repeater (e.g., repeater 140) may be configurable to direct, route, filter, append, insert, amend, and/or delete information (e.g., information on which physical and/or logical direction and/or route the communication signal received from a portable device should be transmitted or directed or routed), including information about itself and/or information about one or more other repeaters, portable devices, caregiver devices, and/or controllers, so as to enable the communication signal received from a portable device (e.g., portable device 110), another repeater (e.g., repeater 140), and/or another controller (e.g., controller 120) to be routed, directed, and/or transmitted properly to the controller (e.g., controller 120) (or appropriate controller). Each repeater (e.g., repeater 140) may also be configurable to direct, route, filter, append, insert, amend, and/or delete location-based and/or directional-based information, including information about a location of itself and/or information obtained through the assistance of one or more other repeaters (e.g., triangulation of signals, best route, backup route in the event of a partial failure, etc.), for or to enable further processing by the controller (e.g., controller 120). Each repeater (e.g., repeater 140) may also be configurable to repeat and/or regenerate a communication signal received from a portable device (e.g., portable device 110) and/or another repeater (e.g., repeater 140) so as to enable the communication signal to arrive at and be processable by the controller (e.g., controller 120).

The controller (e.g., controller 120) may also be in communication with one or more networks (e.g., network 150), such as a Wi-Fi network, Bluetooth network, the internet, one or more intranets, one or more VPNs, mesh-type networks, peer-to-peer networks, client-server networks, etc. Each network (e.g., network 150) may include one or more elements of the system (e.g., system 100) including, but not limited to, one or more controllers (e.g., controller 120), one or more repeaters (e.g., repeater 140), one or more other portable devices (e.g., portable device 110), one or more caregiver devices (e.g., caregiver device 120a), one or more databases (e.g., database 130), and/or one or more graphical displays (e.g., graphical displays 122).

The controller (e.g., controller 120) may also be in communication with one or more graphical displays (e.g., graphical display 122) so as to display notification messages, including information about a user of a portable device (e.g., portable device 110) that has performed a predetermined action, location of the portable device (e.g., portable device 110) and/or user, timestamp (as described in the present disclosure), and/or information about one or more particular caregivers and/or backup caregivers assigned to provide care to the user of the portable device (e.g., portable device 110). Alternatively or in addition, the controller (e.g., controller 120) may also be in communication with one or more other notification devices (not shown), each notification device configurable to provide a visual, audible, and/or physical indication that a portable device (e.g., portable device 110) and/or user of a portable device (e.g., portable device 110) has performed a predetermined action (e.g., detection of a movement of the user or other predetermined actions, as described above and in the present disclosure that are detectable by the sensor). As a non-limiting example, the notification device may be configurable to provide a verbal message indicating information about the user of the portable device (e.g., portable device 110) that has performed the predetermined action, information about the portable device (e.g., portable device 110), information about the location of the portable device (e.g., portable device 110) and/or user, timestamp (as described in the present disclosure), and/or information about one or more caregivers and/or backup caregivers assigned to provide care to the user of the portable device (e.g., portable device 110).

The controller (e.g., controller 120) may also be in communication with one or more caregiver devices (e.g., caregiver device 120a) so as to provide, among other things, such notification messages. In example embodiments, the caregiver device (e.g., caregiver device 120a) may be a mobile phone, PDA, wearable device, tablet, or the like. For example, a caregiver device (e.g., caregiver device 120a) may receive (or receive as priority or marked as priority) notification messages pertaining to users of the portable device (e.g., portable device 110) under their care and/or users that may fall under their care in the event that an assigned caregiver is unavailable or unresponsive. The controller (e.g., controller 120) may also be in communication with one or more other controllers (e.g., controller 120′) in example embodiments.

The controller (e.g., controller 120) may be configurable to perform a processing of information and/or other predetermined actions. The information processed or processable by the controller (e.g., controller 120) may be any information indicative and/or confirming a certain movement (or non-movement) of the portable device (e.g., portable device 110), one or more elements of the portable device (e.g., portable device 110), and/or user of the portable device (e.g., portable device 110). As a non-limiting example, the information processed or processable by the controller (e.g., controller 120) may be information pertaining to an action performed on at least one portion of the sensor assembly (e.g., sensor assembly 112) that indicates, detects, measures, and/or confirms a specific action (or inaction) performed by the user of the portable device (e.g., portable device 110). The specific action (or inaction) performed by the user of the portable device (e.g., portable device 110) may include a specific move (e.g., sensor assembly may be placed on one or more parts of the user's body), sitting up (e.g., sensor assembly may be placed on a buttock and/or back side of the user's body), standing up (e.g., sensor assembly may be placed on a bottom of one or both feet of the user), falling down (e.g., sensor assembly may be placed on one or both knees of the user, one or both palms of the user, and/or on a buttock and/or back side of the user's body), walking (e.g., sensor assembly may be placed on a bottom of one or both feet of the user), jogging (e.g., sensor assembly may be placed on a bottom of one or both feet of the user), and/or running (e.g., sensor assembly may be placed on a bottom of one or both feet of the user). Other user actions are also contemplated without departing from the teachings of the present disclosure.

As another non-limiting example, the information processed or processable by the controller (e.g., controller 120) may include information pertaining to an applied force on at least one portion of the sensor assembly (e.g., sensor assembly 112) that confirms the specific action (or inaction), as described above and in the present disclosure, performed by the user of the portable device (e.g., portable device 110). As another non-limiting example, the information processed or processable by the processor (e.g., processor 116) may include information pertaining to an applied force on the sensor (e.g., sensor 112a) and/or switch (e.g., switch 112b) that indicates, detects, measures, and/or confirms the specific action (or inaction), as described above and in the present disclosure, performed by the user of the portable device (e.g., portable device 110). As another non-limiting example, the information processed or processable by the processor (e.g., processor 116) may be information pertaining to a singular movement or sequence of movements that indicates, detects, measures, and/or confirms the specific action (or inaction), as described above and in the present disclosure, performed by the user of the portable device (e.g., portable device 110).

The Repeater (e.g., Repeater 140).

In an example embodiment, the system (e.g., system 100) may also comprise one or more repeaters (e.g., repeater 140). Each repeater (e.g., repeater 140) may be configurable to communicate with one or more elements of the system (e.g., system 100). As a non-limiting example, each repeater (e.g., repeater 140) may be configurable to communicate, including receive and transmit information, with other repeaters (e.g., repeater 140), one or more portable devices (e.g., portable device 110), one or more controllers (e.g., controller 120), one or more databases (e.g., database 130), one or more networks (e.g., network 150), and/or one or more caregiver devices (e.g., caregiver devices 120a). Such communication between each repeater (e.g., repeater 140) and one or more of the elements of the system (e.g., system 100) described above and in the present disclosure may be in one or more of a variety of forms, including, but not limited to, omni-directional broadcasting, directed or directional broadcasting, peer-to-peer communications, mesh-type communications, connected communications, non-connected communications, full-duplex-type communications, half duplex-type communications, etc. As a non-limiting example, a repeater (e.g., repeater 140) may send a communication signal to a controller (e.g., controller 120) via one or more other repeaters (e.g., repeater 140) and/or one or more portable devices (e.g., portable device 110), all of which may be configured or configurable to be in a broadcast-type, non-connected, and/or half-duplex mesh network.

In an example embodiment, as described in the present disclosure, each repeater (e.g., repeater 140) may be configurable to receive the code and/or a communication signal having the code from a portable device (e.g., portable device 110) and/or another repeater (e.g., repeater 140), and subsequently send (or re-send, forward, direct, transmit) the code and/or communication signal having the code to another repeater (e.g., repeater 140 located physically and/or logically nearer to the controller 120), the controller (e.g., controller 120, such as when there are no repeaters and/or portable devices located physically and/or logically nearer to the controller 120), and/or another portable device (e.g., portable device 110, such as when such portable device is located physically and/or logically nearer to the controller 120). The repeater (e.g., repeater 140) may be further configurable to append information to the code and/or communication signal having the code. Such information may include, for example, an identification of the repeater (e.g., repeater 140), an identification of an intended recipient (e.g., another repeater 140 located physically and/or logically nearer to the controller 120), location-based information, a direction code, a counter (such as a dynamic counter that ensures a signal is sent in one or more specific physical and/or logical directions), and/or other information that enables the code and/or communication signal having the code to be sent (or re-sent, forwarded, directed, transmitted) in one or more specific physical and/or logical directions. It is recognized in the present disclosure that such appending of information by the repeater (e.g., repeater 140) may also assist or enable the controller (e.g., controller 120) to determine a location of the repeater (e.g., repeater 140), portable device (e.g., portable device 110), and/or user of the portable device (e.g., portable device 110).

Alternatively or in addition, the repeater (e.g., repeater 140) may also include a location-based assembly (not shown). The location-based assembly may be configurable to determine a location of the repeater (e.g., repeater 140). As a non-limiting example, the location-based assembly may include a global positioning system (GPS), or the like, configurable to determine a location of the repeater (e.g., repeater 140). As another non-limiting example, the location-based assembly may include functionality to perform, or assist in performing, a triangulation of signals between one or more elements of the system (e.g., system 100), such as one or more other repeaters (e.g., repeater 140), one or more portable devices (e.g., portable device 110), one or more caregiver devices (e.g., caregiver device 120a), one or more elements of or in communication with the network (e.g., network 150) and/or the system (e.g., system 100). In an example embodiment, the code and/or communication signal having the code sent (or re-sent, forwarded, directed, transmitted) by the repeater (e.g., repeater 140) may include, among other things, the location of the repeater (e.g., repeater 140), as determined by the location-based assembly. Furthermore, as described in the present disclosure, a notification message displayed, on the graphical display, may include the location of the repeater (e.g., repeater 140), as determined by the location-based assembly.

Method of Detecting a Movement of One or More Users (e.g., Method 200).

FIG. 7 illustrates an example embodiment of a method (e.g., method 200) of detecting a movement of one or more users. The method may include providing one or more portable devices (e.g., action 202). The portable device (e.g., portable device 110) may be configurable to detect, among other things, a movement of a user. Such detecting may be performable via a sensor (e.g., sensor 112a), switch (e.g., switch 112b), and/or processor (e.g., processor 116). The detecting may include comparing a measurement indicative of the movement to a value, such as the cutoff value described above and in the present disclosure and/or another value. The portable device (e.g., portable device 110) may be attached, secured, and/or coupled to a portion of the user, such as a portion of a foot of the user. More specifically, the sensor assembly (e.g., sensor assembly 112) may be secured firmly to and/or held firmly in place on, such as via a medical adhesive tape 111, or the like, a bottom portion of a foot of a user, as illustrated in FIG. 1. Furthermore, the processor (e.g., processor 116) may be secured firmly to and/or held firmly in place on, such as via an adhesive tape 115, Velcro 115, safety pin 115, clip 115, strap 115, or the like, an article of clothing or lower leg of the user, as illustrated in FIG. 1.

Responsive to the portable device (e.g., portable device 110) detecting a movement, the method (e.g., method 200) may include performing, by the portable device (e.g., portable device 110), a first measurement indicative of the movement (e.g., action 204).

Responsive to the portable device (e.g., portable device 110) detecting a movement, the method (e.g., method 200) may include comparing, by the portable device (e.g., portable device 110), the first measurement to the threshold value, as described above and in the present disclosure, or another value (e.g., action 206). For example, the comparing may be performed by the processor (e.g., processor 116).

Responsive to the portable device (e.g., portable device 110) detecting a movement, the method (e.g., method 200) may include transmitting, by the portable device (e.g., portable device 110), a code when the portable device (e.g., portable device 110) determines, based on the comparing, that the first measurement is greater than or equal to the threshold value, as described above and in the present disclosure, or another value (e.g., action 208). The code may include an identification of the portable device (e.g., portable device 110) and/or the user of the portable device (e.g., portable device 110). For example, the transmitting of the code may be in the form of a communication signal, or the like, and transmitted by the processor (e.g., processor 116). In example embodiments, the code and/or communication signal may be encrypted and/or readable only by an element of the system (e.g., system 100).

The method (e.g., method 200) may further include receiving, by a controller (e.g., controller 120), one or more communication signals, including a first communication signal having the code (e.g., action 210). For example, after the portable device (e.g., portable device 110) transmits the code in the form of a communication signal, the controller (e.g., controller 120) may be configurable to receive the communication signal, either directly from the portable device (e.g., portable device 110) and/or via one or more other elements of the system (e.g., system 100), such as via one or more repeaters (e.g., repeater 140), other portable devices (e.g., portable device 110), network (e.g., network 150), and/or caregiver devices (e.g., caregiver devices 120a).

Responsive to receiving the first communication signal, the method (e.g., method 200) may include obtaining, by the controller (e.g., controller 120), the code from the received first communication signal (e.g., action 212). For example, the controller (e.g., controller 120) may extract and/or replicate the code in the received communication signal. In example embodiments wherein the code and/or communication signal is encrypted, the controller (e.g., controller 120) may be configurable to decrypt the code and/or communication signal.

Responsive to receiving the first communication signal, the method (e.g., method 200) may include retrieving, from a database (e.g., database 130) and based on the obtained code, user information associated with the obtained code (e.g., action 214). The retrieved user information may include an identification of the user of the portable device (e.g. portable device 110) (and/or identification of the portable device). The retrieved user information may also include an identification of a particular one or more caregivers assigned to provide care for the user of the portable device (e.g., portable device 110) based on one or more criteria. For example, the retrieved user information may include an identification of a particular caregiver assigned to provide care for the user of the portable device (e.g., portable device 110) for a specific time or period (e.g., the time the controller receives the communication signal, the time the processor sends the communication signal, etc.), and may also include an identification of another caregiver assigned to be a backup caregiver.

Responsive to receiving the first communication signal, the method (e.g., method 200) may include displaying, on a graphical display (e.g., graphical display 122), a notification message (e.g., action 216). Alternatively or in addition, the method (e.g., method 200) may also include conveying, such as on a speaker (e.g., speaker 122) or other notification forms (e.g., projector, etc.), the notification message. The notification message may include at least the retrieved identification of the user of the portable device (e.g., portable device 110). The notification message may also include the identity of the caregiver and/or backup caregiver assigned to provide care for the user of the portable device (e.g., portable device 110).

### Example 1

As illustrated in FIG. 6, an example embodiment of the system 100 may include a controller 120 and a portable device 110. The system 100 may also include a database 130 and a graphical display 122. In respect to the portable device 110, the portable device 110 may include a sensor 112 and a processor 116, as described above and in the present disclosure. The system 100 may also include a repeater 140, which may be a repeater physically and/or logically nearest to the portable device 110. The repeater 140 may be configurable to communicate with the portable device 110 (and other portable devices (not shown) within the range of the repeater 140) in example embodiments.

The system 100 may also include a second portable device 110′ having a second sensor 112′ and second processor 116′, as described above and in the present disclosure. The system 100 may also include a second repeater 140′, which may be a repeater physically and/or logically further away from the controller 120 as compared to the repeater 140. The second repeater 140′ may be physically and/or logically nearest to the second portable device 110′, and may be configurable to communicate with the second portable device 110′ (and other portable devices (not shown) within the range of the second repeater 140′) in example embodiments. The second repeater 140′ may also be configurable to communicate with the controller 120 and the repeater 140, as well as other repeaters (not shown) within the range of the second repeater 140′.

The system 100 may further include a third portable device 110″ having a third sensor 112″ and third processor 116″, as described above and in the present disclosure. The system 100 may include a third repeater 140″, which may be a repeater physically and/or logically furthest away from the controller 120 as compared to the repeater 140 and second repeater 140′. The third repeater 140″ may be physically and/or logically nearest to the third portable device 110″, and may be configured to communicate with the third portable device 110″ (and other portable devices (not shown) within the range of the third repeater 140″) in example embodiments. The third repeater 140″ may also be configurable to communicate with the repeater 140, as well as other repeaters (not shown) within the range of the third repeater 140″.

In an example situation, the sensor 112 of the portable device 110 may be a force sensor 112 provided to a user of the portable device 110 in a manner similar to that shown in FIG. 2. The force sensor 112 may be configurable to detect an applied force (or impact) on the force sensor 112. When the force sensor 112 detects an applied force indicative of a movement of the user of the portable device 110, the force sensor 112 may perform a measurement of the applied force. When the applied force measurement is greater than or equal to a value, such as the cutoff value, the force sensor 112 may be configurable to provide the applied force measurement to the processor 116. The processor 116 may be configurable to receive the applied force measurement from the force sensor 112. Upon receiving the applied force measurement, the processor 116 may be configurable to compare the applied force measurement to a value, such as the threshold value. The processor 116 may be further configurable to transmit (e.g., broadcast) a signal 160d having a code. The code may be an identification of the portable device 110 and/or the user of the portable device 110, as described above and in the present disclosure.

As illustrated in FIG. 6, the repeater 140 may be configurable to scan for and/or receive signals, including the signal 160d. The repeater 140 may, in turn, transmit (e.g., broadcast) or send (or resend) the signal as signal 160b to the second repeater 140′. The repeater 140 may also be configurable to transmit or send (or resend) the signal as signal 160c to the third repeater 140″ (and to other repeaters and/or portable devices). Upon receiving the signal 160b from the repeater 140, the second repeater 140′ may be configurable to send (or resend) the signal as signal 160a to the controller 120. The signal 160a may include the code. The controller 120, upon receiving the signal 160a, may be configurable to obtain the code from the received signal. The controller 120 may be further configurable to retrieve, from the database and based on the obtained code, user information associated with the code. The retrieved user information may include an identification of the user of the portable device 110. The retrieved user information may also include an identification of the caregiver and/or backup caregiver of the user of the portable device 110. The controller 120 may be further configurable to display a notification message on the graphical display 122, including at least the identification of the user of the portable device 110, identification of the caregiver, identification of the backup caregiver, location of the user of the portable device 110 (e.g., location of the repeater 140), and/or timestamp (e.g., date/time of the detection of the applied force, transmission of the signal 160d, receipt of the signal 160d by the repeater 140, transmission of the signal 160b from the repeater 140, receipt of the signal 160b by the second repeater 140′, transmission of the signal 160a from the second repeater 140′, and/or receipt of the signal 160a by the controller 120).

In an example embodiment, the system 100 may also be configured in such a way that the third repeater 140″ may, upon receiving the signal 160c from the repeater 140, send the signal to another repeater (e.g., if such repeater is physically and/or logically closer or equally as close to the controller 120 than the repeater 140) or not send (or resend) the signal to another repeater (e.g., if no repeaters are physically and/or logically closer or equally as close to the controller 120 than the repeater 140). For example, in situations wherein the system 100 is configured so as to reduce and/or avoid circular transmissions of signals among/between repeaters, the third repeater 140″ may, upon receiving the signal 160c from the repeater 140, not send (or resend) the signal to another repeater (such as to a repeater further away from the controller 120 than the third repeater 140″). This may be accomplished in one of several ways. For example, upon receiving the signal 160d from the portable device 110, the repeater 140 may append to the signal an identification of the repeater 140 (and/or identification of the next repeater 140′ that should receive the signal, direction where the repeater 140 should send, etc.). In this regard, once the third repeater 140″ receives the signal 160c, the third repeater 140″ may be configurable to identify that the signal 160c is not intended for the third repeater 140″ and therefore not send (or resend) the signal.

In an example embodiment for the above situation, if the user of the portable device 110 continues movement (e.g., walks) to another location (e.g., to a location closer to the second repeater 140′ than repeater 140, such as the location of the second portable device 110′ shown in FIG. 6), the portable device 110 may continue to transmit a signal having the code, such as signal 162d. As the second repeater 140′ receives the signal 162d, the second repeater 140′ may be configurable to append to the signal 162d an identification of the second repeater 140′ in a similar manner as described above for the repeater 140. The second repeater 140′ may be configurable to send (or resend) the signal as signals 162a and 162b. The controller 120, upon receiving the signal 162a, may be configurable to obtain the code from the received signal. The controller 120 may be further configurable to retrieve, from the database and based on the obtained code, user information associated with the code. The retrieved user information may include an identification of the user of the portable device 110. The retrieved user information may also include an identification of the caregiver and/or backup caregiver of the user of the portable device 110. The controller 120 may be further configurable to display a second notification message on the graphical display 122, including at least the identification of the user of the portable device 110, identification of the caregiver, identification of the backup caregiver, second location of the user of the portable device 110 (e.g., location of the second repeater 140′), and/or second timestamp (e.g., date/time of the detection of the applied force, transmission of the signal 162d, receipt of the signal 162d by the second repeater 140′, transmission of the signal 162b from the second repeater 140′, and/or receipt of the signal 162a by the controller 120). In an example embodiment, the graphical display 122 may now show two notification messages in time sequence, namely the first notification message having the first time stamp and/or first location, as well as the second notification message having the second time stamp and/or second location.

It is to be understood in the present disclosure that the above example of system 100 may also include one or more elements being transitionable between an off state and an on state, as described above and in the present disclosure. For example, the portable device 100 may also include a switch 112b. The switch 122b, upon receiving the applied force, may be configurable to allow energy source 119 to supply current (or voltage or power) to sensor 112a and/or processor 116, as described above and in the present disclosure. In example embodiments wherein the portable device 100 does not include a switch 112b, the sensor 112a, upon receiving the applied force, may be configurable to allow energy source 119 to supply current (or voltage or power) to the processor 116, as described above and in the present disclosure. It is recognized in the present disclosure that such transitionability between an off state and an on state for certain elements of the system 100, including the sensor 112b and/or processor 116, may enable the portable device 110 to conserve energy and/or increase operational life before an exhaustion of the energy source 119.

### Example 2

As illustrated in FIG. 7, another example embodiment of a system 100 may include controller 120 and portable device 110, the portable device 110 having a sensor 112 and processor 116. The system 100 may also include second portable device 110′ having second sensor 112′ and second processor 116′. The system 100 may also include third portable device 110″ having third sensor 112″ and third processor 116″. The system 100 may also include repeater 140, second repeater 140′, and third repeater 140″. The system 100 may also include fourth repeater 140′″. The system 100 may also include database 130 and graphical display 122.

Similar to the example embodiment described in Example 1, the portable device 110 may be configurable to transmit signal 160d having a code upon detecting, via sensor 112, and processing, via processor 116, a movement of the user of the portable device 100. The signal 160d may be received by repeater 140, and a signal 160c may be sent from repeater 140 to second repeater 140′. Signal 160e may also be sent from repeater 140 to fourth repeater 140′″, and the fourth repeater 140′″ may be configured to not send any further messages when it determines that the signal should be sent towards the controller 120 via second repeater 140′ and not itself, as described above and in the present disclosure. The signal 160c may be received by second repeater 140′, and upon receipt by second repeater 140′, the signal 160b may be sent by second repeater 140′. The signal 160b may be received by the third repeater 140″, and upon receipt by the third repeater 140″, the signal 160a may be sent by the third repeater 140″ to the controller 120. The controller 120 may be configured to obtain the code from the signal 160a and display a notification message on the graphical display 122 in the same manner as described above and in the present disclosure.

In a similar manner, second portable device 110′ may be configurable to detect a movement and send signal 162d having a second code, which results in signals 162c and 162b. Signal 162b may be sent from second repeater 140′ to third repeater 140″, and third repeater 140″ may send signal 162a to the controller 120. The controller 120 may also be configured to obtain the second code from the signal 162a and display a notification message on the graphical display 122 in the same manner as described above and in the present disclosure.

In a similar manner, third portable device 110″ may be configurable to detect a movement and send signal 164d having a third code, which results in signals 164c and 164b. Signal 164b may be sent from third repeater 140″ to the controller 120. The controller 120 may also be configured to obtain the third code from the signal 164a and display a notification message on the graphical display 122 in the same manner as described above and in the present disclosure.

As described above in Example 1 and in the present disclosure, the system 100 may be configurable to continue detecting movements of a user of portable device 110, movements of a user of second portable device 110′, and/or movements of a user of third portable device 110″. Such detecting may be achievable by, for example, the physically and/or logically nearest or closest repeater (e.g., repeater 140, second repeater 140′, third repeater 140″, and/or fourth repeater 140′″) appending information, such as a location-based code and/or repeater identification, to a signal received from such portable device (e.g., portable device 110, second portable device 110′, and/or third portable device 110″). It is recognized in the present disclosure that such continued monitoring of the user of the portable device enables caregivers to be alerted to a movement of the user of the portable device as well as a location (or locations, if the user continues to move) of the user of the portable device.

While various embodiments in accordance with the disclosed principles have been described above, it should be understood that they have been presented by way of example only, and are not limiting. Thus, the breadth and scope of the example embodiments described in the present disclosure should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

For example, as referred to herein, a controller, processor, and/or device may be any computing device or communication device, and may include a virtual machine, computer, node, instance, host, or machine in a networked computing environment. Also as referred to herein, a network or cloud may be a collection of machines connected by communication channels that facilitate communications between machines and allow for machines to share resources. Network may also refer to a communication medium between processes on the same machine. Also as referred to herein, a network element, node, or server may be a machine deployed to execute a program operating as a socket listener and may include software instances.

For example, “assembly”, “apparatus”, “portion”, “segment”, “member”, “body”, “section”, “subsystem”, “system”, or other similar terms should generally be construed broadly to include one part or more than one part attached or connected together.

Memory (or storage or database) may comprise any collection and arrangement of volatile and/or non-volatile components suitable for storing data. For example, memory may comprise random access memory (RAM) devices, read-only memory (ROM) devices, magnetic storage devices, optical storage devices, and/or any other suitable data storage devices. In particular embodiments, memory may represent, in part, computer-readable storage media on which computer instructions and/or logic are encoded. Memory may represent any number of memory components within, local to, and/or accessible by a processor.

Various terms used herein have special meanings within the present technical field. Whether a particular term should be construed as such a “term of art” depends on the context in which that term is used. For example, “connect”, “connected”, “connecting”, “connectable”, “attach”, “attached”, “attaching”, “attachable”, “secure”, “secured”, “securing”, “securable”, “couple”, “coupled”, “coupling”, “in communication with”, “communicating with”, “associated with”, “associating with”, or other similar terms should generally be construed broadly to include situations where attachments, connections, installations, and anchoring are direct between referenced elements or through one or more intermediaries between the referenced elements. As another example, “un-connect,” “un-connected”, “un-connecting”, “un-connectable”, “un-attach”, “un-attached”, “un-attaching”, “un-attachable”, “un-secure”, “un-secured”, “un-securing”, “un-securable”, “uncouple”, “uncoupled”, “uncoupling”, or other similar terms should generally be construed broadly to include situations where separation, removal, and detaching are direct between referenced elements or from one or more intermediaries between the referenced elements. These and other terms are to be construed in light of the context in which they are used in the present disclosure and as one of ordinary skill in the art would understand those terms in the disclosed context. The above definitions are not exclusive of other meanings that might be imparted to those terms based on the disclosed context.

Words of comparison, measurement, and timing such as “at the time”, “equivalent”, “during”, “complete”, and the like should be understood to mean “substantially at the time”, “substantially equivalent”, “substantially during”, “substantially complete”, etc., where “substantially” means that such comparisons, measurements, and timings are practicable to accomplish the implicitly or expressly stated desired result.

Additionally, the section headings and topic headings herein are provided for consistency with the suggestions under various patent regulations and practice, or otherwise to provide organizational cues. These headings shall not limit or characterize the embodiments set out in any claims that may issue from this disclosure. Specifically, a description of a technology in the “Background” is not to be construed as an admission that technology is prior art to any embodiments in this disclosure. Furthermore, any reference in this disclosure to “invention” in the singular should not be used to argue that there is only a single point of novelty in this disclosure. Multiple inventions may be set forth according to the limitations of the claims issuing from this disclosure, and such claims accordingly define the invention(s), and their equivalents, that are protected thereby. In all instances, the scope of such claims shall be considered on their own merits in light of this disclosure, but should not be constrained by the headings herein.

What is claimed is:

1. A system for detecting a movement of users, the system comprising:
a portable device, the portable device having:
a sensor assembly, the sensor assembly having a sensor configurable to perform a first measurement, the sensor transitionable between an off state and an on state, the on state of the sensor being a state resulting when a predetermined action is performed on at least one portion of the sensor assembly, the off state of the sensor being a state resulting when the predetermined action is not performed on the at least one portion of the sensor assembly, wherein the sensor is configured to perform the first measurement when the sensor is in the on state, and wherein the sensor is configured to not perform the first measurement when the sensor is in the off state; and
a processor, the processor configurable to:
compare the first measurement to a threshold value; and
transmit a code when the processor determines, based on the compare, that the first measurement is greater than or equal to the threshold value, the code including an identification of the portable device and/or the user of the portable device;
a database, the database configurable to store information, including the code and user information associated with the code; and
a controller in communication with the database, the controller configurable to:
receive communication signals, including a first communication signal having the code;
obtain, from the received first communication signal, the code;
retrieve, from the database and based on the obtained code, the user information associated with the obtained code, the retrieved user information including an identification of the user of the portable device; and
display, on a graphical display, a notification message, the notification message including at least the retrieved identification of the user of the portable device.

2. The system of claim 1,
wherein the sensor is transitionable between an off state and an on state, the on state being a state resulting when a predetermined action is performed on at least one portion of the sensor assembly, the off state being a state resulting when the predetermined action is not performed on the at least one portion of the sensor assembly; and
wherein the sensor is configurable to perform the first measurement when the sensor is in the on state.

3. The system of claim 1,
further comprising an energy source;
wherein the sensor assembly further comprises a switch in communication with the energy source;
wherein the at least one portion of the sensor assembly receiving the predetermined action includes the switch;
wherein the predetermined action performed on the at least one portion of the sensor assembly includes an application of a force, on the switch, greater than or equal to a cutoff value;
wherein the first measurement is a measurement of a force applied to the sensor;
wherein the cutoff value is less than the threshold value;
wherein, when the predetermined action is performed on the switch, the switch is configurable to allow current to pass from the energy source to the sensor so as to enable the sensor to perform the first measurement; and
wherein, when the predetermined action is not performed on the switch, the switch is configurable to prevent current from passing from the energy source to the sensor so as to disable the sensor from performing the first measurement.

4. The system of claim 3,
further comprising an energy source in communication with the switch;
wherein, when the predetermined action is performed on the switch, the switch is configurable to allow current to pass from the energy source to the sensor so as to enable the sensor to perform the first measurement; and
wherein, when the predetermined action is not performed on the switch, the switch is configurable to prevent current from passing from the energy source to the sensor so as to disable the sensor from performing the first measurement.

5. The system of claim 1,
further comprising an energy source in communication with the sensor;
wherein the at least one portion of the sensor assembly receiving the predetermined action includes the sensor;
wherein the predetermined action performed on the at least one portion of the sensor assembly includes an application of a force, on the sensor, greater than or equal to a cutoff value;
wherein the first measurement is a measurement of the force applied to the sensor;
wherein the cutoff value is less than the threshold value;
wherein, when the predetermined action is performed on the sensor, the sensor is configurable to allow current to pass from the energy source to the sensor so as to enable the sensor to perform the first measurement; and
wherein, when the predetermined action is not performed on the sensor, the sensor is configurable to prevent current from passing from the energy source to the sensor so as to disable the sensor from performing the first measurement.

6. The system of claim 5,
further comprising an energy source in communication with the sensor;
wherein, when the predetermined action is performed on the sensor, the sensor is configurable to allow current to pass from the energy source to the sensor so as to enable the sensor to perform the first measurement; and
wherein, when the predetermined action is not performed on the sensor, the switch is configurable to prevent current from passing from the energy source to the sensor so as to disable the sensor from performing the first measurement.

7. The system of claim 1, wherein one or more of the following apply:
the predetermined action performed on the at least one portion of the sensor assembly includes a receiving, by the sensor, of a sequence of movements;
the first measurement is a collection of measurements indicative of the sequence of movements;
the threshold value is configurable based on a predetermined sequence of movements of the portable device indicative of the user walking;
the processor is transitionable between an off state and an on state, the on state being a state resulting when a predetermined action is performed on at least one portion of the sensor assembly, the off state being a state resulting when the predetermined action is not performed on the at least one portion of the sensor assembly; and/or
the processor is configurable to perform the compare and transmit when the processor is in the on state.

8. The system of claim 1,
wherein the processor is transitionable between an off state and an on state, the on state being a state resulting when a predetermined action is performed on at least one portion of the sensor assembly, the off state being a state resulting when the predetermined action is not performed on the at least one portion of the sensor assembly; and
wherein the processor is configurable to perform the compare and transmit when the processor is in the on state.

9. The system of claim 8,
wherein the sensor assembly further comprises a switch;
wherein the at least one portion of the sensor assembly receiving the predetermined action includes the switch;
wherein the predetermined action performed on the at least one portion of the sensor assembly includes an application of a force, on the switch, greater than or equal to a cutoff value;
wherein the first measurement is a measurement of a force applied to the sensor; and
wherein the cutoff value is less than the threshold value.

10. The system of claim 9,
further comprising an energy source in communication with the switch;
wherein, when the predetermined action is performed on the switch, the switch is configurable to allow current to pass from the energy source to the processor so as to transition the processor to the on state; and
wherein, when the predetermined action is not performed on the switch, the switch is configurable to prevent current from passing from the energy source to the processor so as to maintain the processor in the off state.

11. The system of claim 8,
wherein the at least one portion of the sensor assembly receiving the predetermined action includes the sensor;
wherein the predetermined action performed on the at least one portion of the sensor assembly includes an application of a force, on the sensor, greater than or equal to a cutoff value;
wherein the first measurement is a measurement of the force applied to the sensor; and
wherein the cutoff value is less than the threshold value.

12. The system of claim 11,
further comprising an energy source in communication with the sensor;
wherein, when the predetermined action is performed on the sensor, the sensor is configurable to allow current to pass from the energy source to the processor so as to transition the processor to the on state;
wherein, when the predetermined action is not performed on the sensor, the sensor is configurable to prevent current from passing from the energy source to the processor so as to maintain the processor in the off state.

13. The system of claim 8,
wherein the predetermined action performed on the at least one portion of the sensor assembly includes a receiving, by the sensor, of a sequence of movements;
wherein the first measurement is a collection of measurements indicative of the sequence of movements; and
wherein the threshold value is configurable based on a predetermined sequence of movements of the portable device indicative of the user walking.

14. The system of claim 1, wherein one or more of the following apply:
the controller is further configurable to transmit, to a caregiver mobile device, the notification message;
the portable device further includes a location-based assembly configurable to determine a location of the portable device;
the code transmitted by the processor includes the location of the portable device;
the notification message displayed, on the graphical display, includes the location of the portable device; and/or
the controller is configurable to track movement of the portable device based on the location of the portable device received from the processor.

15. The system of claim 1,
wherein the portable device further includes a location-based assembly configurable to determine a location of the portable device;
wherein the code transmitted by the processor includes the location of the portable device; and
wherein the notification message displayed, on the graphical display, includes the location of the portable device.

16. The system of claim 15, wherein the controller is configurable to track movement of the portable device based on the location of the portable device received from the processor.

17. The system of claim 1,
further comprising a plurality of repeaters, including a first repeater and second repeater, each repeater configurable to receive and transmit communication signals;
wherein the first repeater and second repeater are each configurable to communicate with the processor, including receiving the code from the processor;
wherein the first repeater and second repeater are further configurable to determine a location of the processor.

18. The system of claim 1,
further comprising a plurality of repeaters, including a first repeater and a second repeater configured as being closer to the controller than the first repeater, each repeater configurable to receive and transmit communication signals;
wherein, when the first repeater receives the code from the portable device:
the first repeater is configurable to transmit a first signal, the first signal including the code from the portable device and an indication of (i) the first repeater and/or (ii) an intended recipient, wherein the intended recipient includes the second repeater; and
the second repeater, upon receiving the first signal, is configurable to transmit a second signal after confirming that (i) the second repeater is the intended recipient and/or (ii) the first repeater was the sender of the first signal; wherein the second signal includes the code from the portable device and an indication of (i) the second repeater and/or (ii) a second intended recipient, the second intended recipient being the controller.

19. The system of claim 18, wherein, when the first repeater receives the code from the portable device:
the first repeater is further configurable to include, in the first signal, location information indicative of a location of the first repeater;
the second repeater is further configurable to include, in the second signal, the location information indicative of the location of the first repeater; and
the notification message displayed, by the controller, further includes the location information indicative of the location of the first repeater.

20. The system of claim 1,
further comprising a plurality of repeaters, including a distal repeater, a proximal repeater configured as being closer to the controller than the distal repeater, and an intermediate repeater configured as being between the distal repeater and the proximal repeater, each repeater configurable to receive and transmit communication signals;
wherein, when the distal repeater receives the code from the portable device:
the distal repeater is configurable to transmit a first signal, the first signal including the code from the portable device;
the intermediate repeater, upon receiving the first signal, is configurable to re-transmit the first signal to a repeater that is configured as being closer to the controller than the intermediate repeater; and
the proximal repeater, upon receiving the first signal, is configurable to re-transmit the first signal (i) to a repeater that is configured as being closer to the controller than the proximal repeater, or (ii) to the controller if there are no repeater that are configured as being closer to the controller than the proximal repeater.

21. The system of claim 20, wherein, when the distal repeater receives the code from the portable device:
the distal repeater is further configurable to include, in the first signal, location information indicative of a location of the distal repeater; and
the notification message displayed, by the controller, further includes the location information indicative of the location of the distal repeater.

22. The system of claim 1,
further comprising a plurality of repeaters, including a distal repeater, a proximal repeater configured as being closer to the controller than the distal repeater, and an intermediate repeater configured as being between the distal repeater and the proximal repeater, each repeater configurable to receive and transmit communication signals;
wherein, when the distal repeater receives the code from the portable device:
the distal repeater is configurable to transmit a first signal, the first signal including the code from the portable device and an indication of an intended recipient, the intended recipient being the intermediate repeater;
the intermediate repeater, upon receiving the first signal, is configurable to transmit a second signal after confirming that the intermediate repeater is the intended recipient, the second signal including the code from the portable device and an indication of a second intended recipient, the second intended recipient being the proximal repeater; and
the proximal repeater, upon receiving the second signal, is configurable to transmit a third signal after confirming that the proximal repeater is the intended recipient, the third signal including the code from the portable device and an indication of a third intended recipient, the third intended recipient being the controller.

23. The system of claim 22, wherein, when the distal repeater receives the code from the portable device:
the distal repeater is further configurable to include, in the first signal, location information indicative of a location of the distal repeater;
the intermediate repeater is further configurable to include, in the second signal, the location information indicative of the location of the distal repeater;
the proximal repeater is further configurable to include, in the third signal, the location information indicative of the location of the distal repeater; and
the notification message displayed, by the controller, further includes the location information indicative of the location of the distal repeater.

24. The system of claim 1,
further comprising a second portable device, the second portable device having:
a second sensor assembly, the second sensor assembly having a second sensor configurable to perform a second measurement; and
a second processor, the second processor configurable to:
compare the second measurement to a second threshold value; and
transmit a second code when the second processor determines, based on the compare of the second measurement to the second threshold value, that the second measurement is greater than or equal to the second threshold value, the second code including an identification of the second portable device and/or the user of the second portable device;
wherein the controller is further configurable to:
receive a second communication signal having the second code;
obtain, from the received second communication signal, the second code;
retrieve, from the database and based on the obtained second code, the user information associated with the second code, the retrieved user information associated with the second code including an identification of the user of the second portable device; and
display, on the graphical display, a second notification message, the second notification message including at least the retrieved identification of the user of the second portable device; and
wherein the controller is configurable to simultaneously display, on the graphical display, the first notification message and the second notification message.

25. The system of claim 24, wherein the controller is configurable to simultaneously display, on the graphical display, the first notification message and the second notification message.

26. A portable device for detecting a movement of a user, the portable device comprising:
a sensor assembly having a sensor, the sensor configurable to perform a first measurement; and
a processor, the processor transitionable between an off state and an on state, the on state being a state resulting when a predetermined action is performed on at least one portion of the sensor assembly, the off state being a state resulting when the predetermined action is not performed on the at least one portion of the sensor assembly, the processor configurable to:
responsive to the processor being in the on state:
compare the first measurement to a threshold value; and
transmit a signal when the processor determines, based on the compare, that the first measurement is greater than or equal to the threshold value.

27. The portable device of claim 26,
wherein the sensor assembly further comprises a switch; and
wherein the at least one portion of the sensor assembly receiving the predetermined action includes the switch.

28. The portable device of claim 26,
wherein the predetermined action performed on the at least one portion of the sensor assembly includes an application of a force, on the at least one portion of the sensor assembly, greater than or equal to a cutoff value;
wherein the first measurement is a measurement of a force applied to the at least one portion of the sensor assembly; and
wherein the cutoff value is less than the threshold value.

29. The portable device of claim 26,
further comprising an energy source in communication with the at least one portion of the sensor assembly;
wherein, when the predetermined action is performed on the at least one portion of the sensor assembly, the at least one portion of the sensor assembly is configurable to allow current to pass from the energy source to the processor so as to transition the processor to the on state; and
wherein, when the predetermined action is not performed on the at least one portion of the sensor assembly, the at least one portion of the sensor assembly is configurable to prevent current from passing from the energy source to the processor so as to maintain the processor in the off state.

30. The portable device of claim 29,
wherein, when the predetermined action is performed on the at least one portion of the sensor assembly, the at least one portion of the sensor assembly is configurable to allow current to pass from the energy source to the sensor so as to enable the sensor to perform the first measurement; and
wherein, when the predetermined action is not performed on the at least one portion of the sensor assembly, the at least one portion of the sensor assembly is configurable to prevent current from passing from the energy source to the sensor so as to disable the sensor from performing the first measurement.

31. The portable device of claim 26,
wherein the at least one portion of the sensor assembly receiving the predetermined action includes the sensor;
wherein the predetermined action performed on the at least one portion of the sensor assembly includes an application of a force, on the sensor, greater than or equal to a cutoff value;
wherein the first measurement is a measurement of the force applied to the sensor; and
wherein the cutoff value is less than the threshold value.

32. The portable device of claim 31,
further comprising an energy source in communication with the sensor;
wherein, when the predetermined action is performed on the sensor, the sensor is configurable to allow current to pass from the energy source to the processor so as to transition the processor to the on state;
wherein, when the predetermined action is not performed on the sensor, the sensor is configurable to prevent current from passing from the energy source to the processor so as to maintain the processor in the off state.

33. The portable device of claim 26, wherein one or more of the following apply:
the predetermined action performed on the at least one portion of the sensor assembly includes a receiving, by the sensor, of a sequence of movements;
the first measurement is a collection of measurements indicative of the sequence of movements;
the threshold value is configurable based on a predetermined sequence of movements of the portable device indicative of the user walking;
the signal includes a code, the code being an identification of the portable device and/or the user;
the sensor assembly is adaptable to be secured to a bottom portion of the user's feet;
the threshold value is a value based on a weight of the user; and/or
the portable device further comprises a memory assembly in communication with the processor, the memory assembly configurable to store the code and the threshold value.

34. The portable device of claim 26, wherein one or more of the following apply:
the signal includes a code, the code being an identification of the portable device and/or the user;
the sensor assembly is adaptable to be secured to a bottom portion of the user's feet;
the threshold value is a value based on a weight of the user; and/or
the portable device further comprises a memory assembly in communication with the processor, the memory assembly configurable to store the code and the threshold value.

35. A portable device for detecting a movement of a user, the portable device comprising:
a sensor assembly having a sensor, the sensor transitionable between an off state and an on state, the on state being a state resulting when a predetermined action is performed on at least one portion of the sensor assembly, the off state being a state resulting when the predetermined action is not performed on the at least one portion of the sensor assembly, the sensor configurable to:
responsive to the sensor being in the on state:
perform a first measurement; and
a processor in communication with the sensor assembly, the processor configurable to:
receive, from the sensor assembly, the first measurement;
compare the first measurement to a threshold value; and
transmit a signal when the processor determines, based on the compare, that the first measurement is greater than or equal to the threshold value.

36. The portable device of claim 35,
wherein the sensor assembly further comprises a switch; and
wherein the at least one portion of the sensor assembly receiving the predetermined action includes the switch.

37. The portable device of claim 35,
wherein the predetermined action performed on the at least one portion of the sensor assembly includes an application of a force, on the at least one portion of the sensor assembly, greater than or equal to a cutoff value;
wherein the first measurement is a measurement of a force applied to the at least one portion of the sensor assembly; and
wherein the cutoff value is less than the threshold value.

38. The portable device of claim 35,
further comprising an energy source in communication with the at least one portion of the sensor assembly;
wherein, when the predetermined action is performed on the at least one portion of the sensor assembly, the at least one portion of the sensor assembly is configurable to allow current to pass from the energy source to the sensor so as to enable the sensor to perform the first measurement; and
wherein, when the predetermined action is not performed on the at least one portion of the sensor assembly, the at least one portion of the sensor assembly is configurable to prevent current from passing from the energy source to the sensor so as to disable the sensor from performing the first measurement.

39. The portable device of claim 35,
wherein the at least one portion of the sensor assembly receiving the predetermined action includes the sensor;
wherein the predetermined action performed on the at least one portion of the sensor assembly includes an application of a force, on the sensor, greater than or equal to a cutoff value;
wherein the first measurement is a measurement of the force applied to the sensor; and
wherein the cutoff value is less than the threshold value.

40. The portable device of claim 39,
further comprising an energy source in communication with the sensor;
wherein, when the predetermined action is performed on the sensor, the sensor is configurable to allow current to pass from the energy source to the sensor so as to enable the sensor to perform the first measurement; and
wherein, when the predetermined action is not performed on the sensor, the switch is configurable to prevent current from passing from the energy source to the sensor so as to disable the sensor from performing the first measurement.

41. The portable device of claim 35, wherein one or more of the following apply:
the predetermined action performed on the at least one portion of the sensor assembly includes a receiving, by the sensor, of a sequence of movements;
the first measurement is a collection of measurements indicative of the sequence of movements;
the threshold value is configurable based on a predetermined sequence of movements of the portable device indicative of the user walking;
the signal includes a code, the code being an identification of the portable device and/or the user;
the sensor assembly is adaptable to be secured to a bottom portion of the user's feet;
the threshold value is a value based on a weight of the user; and/or
the portable device further comprises a memory assembly in communication with the processor, the memory assembly configurable to store the code and the threshold value.

42. The portable device of claim 35, wherein one or more of the following apply:
the signal includes a code, the code being an identification of the portable device and/or the user;
the sensor assembly is adaptable to be secured to a bottom portion of the user's feet;
the threshold value is a value based on a weight of the user; and/or
the portable device further comprises a memory assembly in communication with the processor, the memory assembly configurable to store the code and the threshold value.

43. A method of detecting a movement of users, the method comprising:
providing a portable device, the portable device configurable to detect a movement of a user;
responsive to the portable device detecting a movement of the portable device:
performing, by the portable device, a first measurement indicative of the movement of the portable device;
comparing, by the portable device, the first measurement to a threshold value; and
transmitting, by the portable device, a code when the portable device determines, based on the comparing, that the first measurement is greater than or equal to the threshold value, the code including an identification of the portable device and/or the user of the portable device;
receiving, by a controller, communication signals, including a first communication signal having the code;
responsive to receiving the first communication signal:
obtaining, by the controller, the code from the received first communication signal;
retrieving, from a database and based on the obtained code, user information associated with the obtained code, the retrieved user information including an identification of the user of the portable device; and
displaying, on a graphical display, a notification message, the notification message including at least the retrieved identification of the user of the portable device.
